(19) United States
(12) Patent Application Publication
(10) Pub. No.: US 2011/0282343 A1
(43) Pub. Date: Nov. 17, 2011

(54) METHOD AND DEVICE FOR TREATMENT OF ARRHYTHMIAS AND OTHER MALADIES

(75) Inventor: Christopher Gerard Kunis, Escondido, CA (US)

(73) Assignee: MEDICAL DEVICE INNOVATIONS LLC, Escondido, CA (US)

(21) Appl. No.: 13/106,343

(22) Filed: May 12, 2011

Related U.S. Application Data
(60) Provisional application No. 61/334,079, filed on May 12, 2010.
(60) Provisional application No. 61/366,855, filed on Jul. 22, 2010.
(60) Provisional application No. 61/390,102, filed on Oct. 5, 2010.
(60) Provisional application No. 61/443,807, filed on Feb. 17, 2011.

(51) Int. Cl. A61B 18/14; A61M 29/00

(52) U.S. Cl. 606/41; 606/191

(57) ABSTRACT

Devices and methods are described for treating maladies such as atrial fibrillation. The devices and methods, in some implementations, include two rings separated by a helical winding. The rings and at least one helical winding provide mechanical pressure against an adjacent tissue, e.g., the tissue of a vessel, and the pressure works to inhibit the propagation of electrical signals along the vessel.

### CROSS-REFERENCE TO RELATED APPLICATIONS

This application claims benefit of priority to the following US provisional patent applications: U.S. Provisional Patent Application Ser. No. 61/334,079, filed May 12, 2010, entitled “Method and Device for Treatment of Arrhythmias and Other Maladies”;U.S. Provisional Patent Application Ser. No. 61/366,855, filed Jul. 22, 2010, entitled “Method and Device for Treatment of Arrhythmias and Other Maladies”;U.S. Provisional Patent Application Ser. No. 61/390,102, filed Oct. 5, 2010, entitled “Method and Device for Treatment of Arrhythmias and Other Maladies”;U.S. Provisional Patent Application Ser. No. 61/443,807, filed Feb. 17, 2011, entitled “Method and Device for Treatment of Arrhythmias and Other Maladies”; all of which are owned by the assignee of the present application and are incorporated by reference herein in their entirety.

### BACKGROUND

Atrial fibrillation is a common and dangerous disease. It is the most common arrhythmia, and accounts for approximately ⅓ of all hospitalizations due to heart rhythm disorders. In addition, atrial fibrillation patients have a greatly increased risk of stroke mortality.

Current first-line therapies for atrial fibrillation include the use of anti-arrhythmic drugs and anti-coagulation agents. Drugs are useful at reducing symptoms, but often include undesirable side effects. Anti-coagulation agents can reduce the risk of stroke, but often increase the risk of bleeding.

Emerging second-line therapies include surgical and catheter ablation. However, the same are associated with high complication rates, long procedure times, and limited clinical evidence. In addition, their administration typically requires extensive training in the use and installation of complex technology.

Pulmonary vein (PV) isolation is the cornerstone of ablation strategies. The same is currently achieved by causing destructive lesions near the PV. Devices for doing so include point-by-point tip catheters as well as cryoablation devices and radiofrequency ablation devices.

### SUMMARY

The present method and device relate to an implanted device that has an improved safety profile and which minimizes collateral damage over current therapies. Therapy is delivered within the vessel having a focal tissue effect sufficient to create electrically inert tissue at the point of contact affecting only the implant deployment location, e.g., where ectopic beats occur within the sleeve of the pulmonary vein. No external energy source or capital investment is required for use with this device. Furthermore, there is no need for 3-D mapping for placement, although mapping may be employed and the same may be provided, e.g., by a delivery device itself. The system and method may be especially suited for treating paroxysmal patients and/or patients who have failed a previous RF ablation where micro-reentrant signals have propagated.

Unlike some prior devices, the device and method need not directly integrate into the wall surface of the PVs to obtain isolation, nor is it necessary to cause injury to the tissue via any means of cutting or scoring of atrial or PV cardiac tissue. Rather, in an acute treatment, the device is designed to apply and maintain radial or substantially radial force along the circumference of the PVs at the ostium, as well as distal to the ostium, while employing a helical pattern of extension arms, connecting the two or more ring-like coils, to disrupt the electrical substrate.

Implementations of the device and method are configured to treat atrial fibrillation without requiring the delivery of energy, without employing needles or other penetrating elements, and without employing elements for scarring. Rather, the device provides mechanical energy against cardiac tissue, e.g., against the intimal lining of the PV, eliminating the electrical refractory process of the myocytes on a cellular level and inhibiting the chemical reaction at the focal site of the implant, thus rendering the tissue electrically inert at the contact point of the implant and creating focal necrosis in a line of block.

The technology may apply mechanical pressure causing a two-step biological response. First, an acute response is caused by pressure-induced apoptosis inhibiting chemical exchange of sodium/calcium and disrupting focal electrical wave propagation. Secondly, a biological response for chronic or long-term isolation/denervation is provided by causing focal endothelial cell proliferation at the implant site. Of course, other processes may also take place, but the above are believed to be important (though these explanations should not be thought of as limiting in any way the scope of the invention).

In one aspect, the invention is directed towards an implant device for treating a malady, including: a proximal ring; a distal ring; and an extension arm connecting the proximal ring to the distal ring.

Implementations of the invention may include one or more of the following. The extension arm may include at least one helical winding. The proximal ring and the distal ring may include coils of a ribbon. The radius of the proximal ring may be greater than the radius of the distal ring. Each coil may include at least one winding of the ribbon, e.g., at least 1.5 windings of the ribbon. Each coil may include a pressure feature such as a ridge. In an undeployed configuration, the radius of the proximal ring may be between about 4 to 60 mm and the radius of the distal ring may be between about 6 to 60 mm. In a deployed configuration, the radius of the proximal ring may be between about 2 to 40 mm and the radius of the distal ring may be between about 3 to 40 mm. The rings may be configured to deliver a force against adjacent tissue when deployed of between about 5 g/mm² and 340 g/mm², e.g., between about 20 g/mm² and 200 g/mm². The proximal ring may be configured to deliver a lesser force when deployed against adjacent tissue than the distal ring. The width of the ribbon may be between about 0.5 and 2.5 mm, e.g., 1 and 2 mm. An extremity of the ring may be shaped to increase frictional or mechanical resistance against movement, e.g., may be shaped to include scallops, ribs, or a club shaped end. The implant device may be coated with a material composition, surface treatment, coating, or biological agent and/or drug.

In another aspect, the invention is directed towards a kit for treating a malady by deploying an implant device in a vessel, including the above-noted implant device, and a delivery system, the delivery system including a catheter having a pigtail distal end, such that upon deployment of the implant device from the pigtail distal end, a longitudinal axis of the implant device is substantially collinear with a longitudinal axis of the vessel.

In another aspect, the invention is directed towards a method for treating a malady, including: choosing a size of an implant device for insertion into a vessel of a patient, the implant device including a proximal ring, a distal ring, and an extension arm connecting the proximal ring to the distal ring; inserting the implant device into the vessel of the patient, such that the choosing includes selecting the size of the implant device such that the implant device compresses a K, Ca, or Na channel in adjacent tissue sufficiently to block or to delay electrical signals traveling along the axis of the vessel.

Implementations of the invention may include one or more of the following. The inserting may include delivering the implant to the vessel through a catheter including a pigtail distal end. The vessel may be a pulmonary vein. The method may further include mapping at least one pulmonary vein and/or ablating at least one pulmonary vein. The ablating may be performed using at least one electrode disposed on a delivery device. The inserting may include delivering the distal ring into the pulmonary vein and delivering the proximal ring into the ostium of the pulmonary vein. The method may further include administering local anesthesia and not general anesthesia to the patient. The inserting may further include pushing the implant device through the catheter with a pushing mechanism or means. The pushing mechanism for means may be coupled to the implant device using a grabbing means. The mapping may include determining the sizes of at least two pulmonary veins, and may further include delivering at least one implant device to each pulmonary vein. The method may further include loading implant devices into the delivery device in the order in which they are to be successively implanted in pulmonary veins. The malady may be atrial fibrillation or vessel non-patency. The method may further include inducing a local heating effect to be present on the implant device by induction. The method may further include recapturing the implant device after the inserting. The compression of the K, Ca, or Na channel in adjacent tissue sufficiently to block electrical signals traveling along the axis of the vessel may include compressing the first one to five cellular layers of the adjacent tissue. The mapping may be performed both before the inserting and after the inserting. The compression may be such that the delay is caused in conduction of at least 50%.

In another aspect, the invention is directed to a method for treating a malady, including: choosing a size of an implant device for insertion into a vessel of a patient, the implant device including a proximal ring, a distal ring, and an extension arm connecting the proximal ring to the distal ring; and inserting the implant device into the vessel of the patient, such that the choosing includes selecting the size of the implant device such that the implant device causes a necrosis in adjacent tissue sufficient to block electrical signals traveling along the axis of the vessel.

In another aspect, the invention is directed to a method for treating a malady, including: choosing a size of an implant device for insertion into a vessel of a patient, the implant device including a proximal ring, a distal ring, and an extension arm connecting the proximal ring to the distal ring; inserting the implant device into the vessel of the patient, such that the choosing includes selecting a radius of the distal ring of the implant device to be at least two times the radius of the vessel.

Implementations of the invention may include one or more of the following. The method may further include selecting a radius of the distal ring of the implant device to be at least five times the radius of the vessel.

In another aspect, the invention is directed to a method for treating a malady, including: inserting a catheter into a vessel of a patient, the catheter having loaded within an anchoring device for partial insertion into a vessel of a patient, the anchoring device including at least a distal ring; partially extending the distal ring from the catheter such that the distal ring is anchored in the vessel; activating at least one electrode on the catheter, the at least one electrode substantially adjacent to tissue when the distal ring is anchored in the vessel, the activating causing ablation and necrosis of the adjacent tissue; retracting the distal ring into the catheter; and withdrawing the catheter.

Implementations of the invention may include one or more of the following. The method may further include activating a plurality of electrodes on the catheter, the electrodes distributed along the pigtail distal end. The method may further include rotating the catheter at least partially during the activating, thereby causing ablation and necrosis of tissue and the creation of partial circumferential linear lesions. The method may further include inserting an implant device into the vessel, the implant device including a proximal ring, a distal ring, and an extension arm between the proximal and distal ring.

Advantages of the invention may include one or more of the following. The device can be deployed into the target zone, e.g., into the PV, where cryoablation and radio frequency ablation techniques cannot. Devices may be employed to provide multiple locations of circumferential block as well as lateral disruption along the PV sleeve to dissociate ectopic beats that emulate from within the PVs. The device may be delivered using a procedure under only local anesthesia rather than requiring general anesthesia.

### BRIEF DESCRIPTION OF THE DRAWINGS

FIG. 1 schematically illustrates an implant device within a vessel, e.g., a pulmonary vein.

FIGS. 2(A)-(C) illustrate various views of the implant device of FIG. 1, with a single helix connecting two coils or rings.

FIGS. 3(A)-(C) illustrate various views of another embodiment of the implant device, illustrating how two helices or a dual helix system may be employed to connect two coils or rings.

FIGS. 4(A)-(B) illustrates features that may be employed in certain implementations of the implant device.

FIG. 5 illustrates a feature that may be employed in certain implementations of the implant device.

FIG. 6 illustrates a feature that may be employed in certain implementations of the implant device.

FIG. 7 illustrates details of a delivery device that may be employed to deliver the implant device.

FIG. 8 illustrates details of the device of FIG. 7.

FIG. 9 illustrates additional details of the device of FIG. 7.

FIG. 10 illustrates a perspective view of the device of FIG. 7.

FIGS. 11(A)-(C) illustrate proximal, distal end, and distal tip details of the device of FIG. 7.

FIG. 12 schematically illustrates an implant device as well as a delivery device that may be used for implantation.

FIG. 13(A) illustrates a terminal end of an implant device, showing the end which may be grabbed by a grabber associated with the delivery device, or with a retrieval device. FIG. 13(B) illustrates the grabber associated with the delivery device, or with a retrieval device.

FIGS. 14(A) and (B) illustrate the grabber device, in both a closed and opened configuration, respectively. FIG. 14(C) illustrates a cutaway view of the grabber device in use within a delivery device.

FIG. 15 illustrates a system having a similar configuration as the implant device but which may be employed to ablate tissue using radio frequencies.

FIGS. 16(A) and (B) illustrate views of another embodiment of the system of FIG. 15. FIG. 16(A) illustrates the device in a vein and FIG. 16(B) illustrates necrosed tissue patterns that may be created.

FIG. 17 illustrates removal of the implant device from a delivery device using a pusher and ratchet sleeve.

FIG. 18 illustrates a ratchet sleeve that may be employed to remove the implant device from a delivery device.

FIGS. 19(A)-(D) illustrate steps in removing the implant device from one embodiment of a delivery device, where the implant device expands off a mandrel.

FIGS. 20(A)-(D) illustrate steps in removing the implant device from another embodiment of a delivery device, where the implant device is pushed out of a tube.

Like reference numerals refer to like elements throughout.

### DETAILED DESCRIPTION

### Implant Device Details

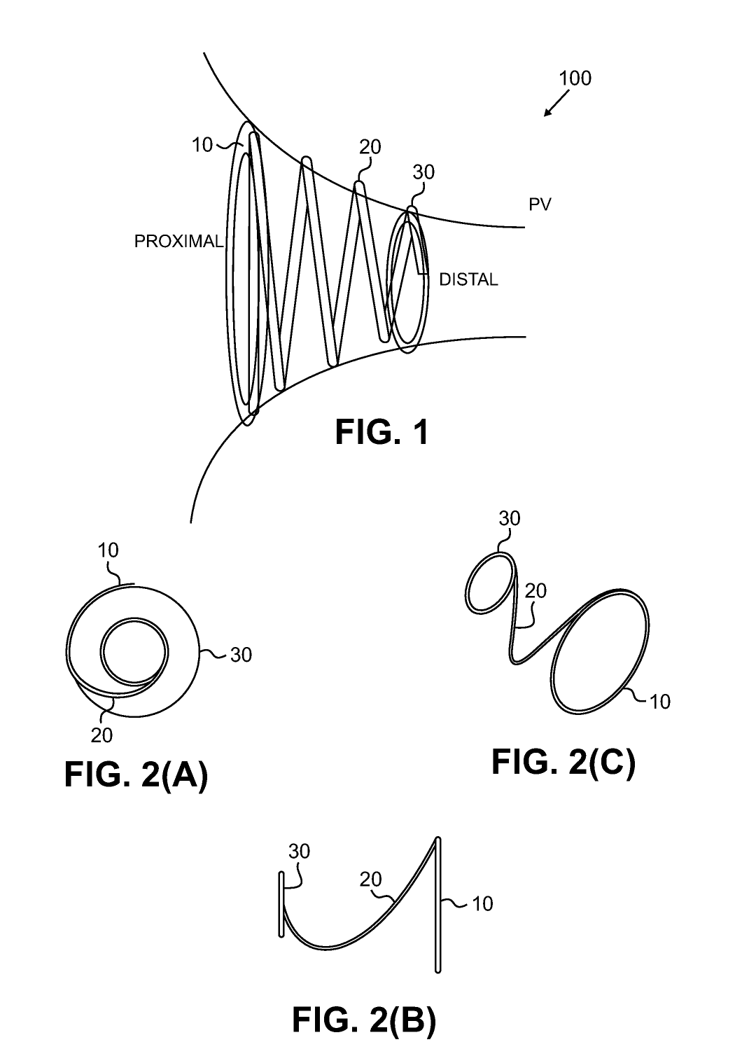

In one implementation, the implant may include two separated coils or rings that are connected by a single helical wire, a double helical wire, or a set of multiple helical wires. Such an implant, in place within a vessel such as the PV, is illustrated schematically in FIGS. 1-3.

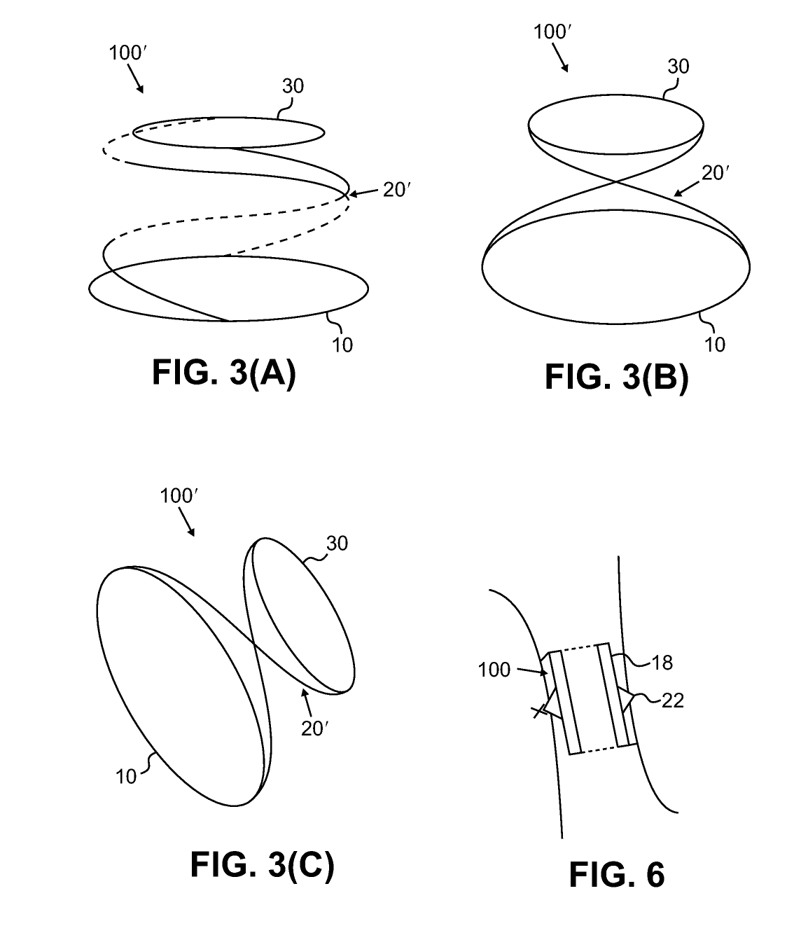

Referring to FIG. 1, an implant device 100 is illustrated schematically within a pulmonary vein. The implant device 100 includes a proximal coil 10, a distal coil 30, and the two are separated by a helix or helical wind 20. FIGS. 2(A)-(C) illustrate various views of the implant device of FIG. 1, where a single helical wind is employed between the coils. FIGS. 3(A)-(C) illustrate the situation where a double helical wind 20′ is employed between coils 10 and 30.

The diameter of the undeployed coils may be about 4 mm to 60 mm for the proximal coil, and about 6 mm to 60 mm for the distal coil. The diameter of the deployed coils may be about 2 mm to 40 mm for the proximal coil, and about 3 mm to 40 mm for the distal coil. The coils may be configured in a symmetrical pattern, e.g., the diameter of the distal coil may be substantially equal to the diameter of the proximal coil. Alternatively, an asymmetric pattern may be employed having one end of the coil larger or smaller then the other end, e.g., a distal end may have a 15 mm diameter while the proximal end may have a larger 25 mm diameter. Using these values, the coils when undeployed may be significantly oversized compared to the vessels for which they are intended. They may be, e.g., oversized by 10-100%, e.g., 20-60%, and good results have been seen also for values of 45-55%, e.g., 50% oversizing.

The rings may be designed to deliver a force against the tissue of between about 5 g/mm² and 340 g/mm², e.g., between about 20 g/mm² and 200 g/mm² The distal ring may provide a greater amount of force than the proximal one.

One or more of the helices may revolve around a central axis 1, 1.5, 2, 3, or more times. In this way, even when placed in larger veins, the available expansion room may cause an effective pressure block to be achieved. However, in this regard, it is noted that radial force decreases dramatically as the radius increases.

For implants made from ribbon wires, exemplary values of the ribbon width may be, e.g., 1-2 mm, e.g., between 0.5 and 2.5 mm.

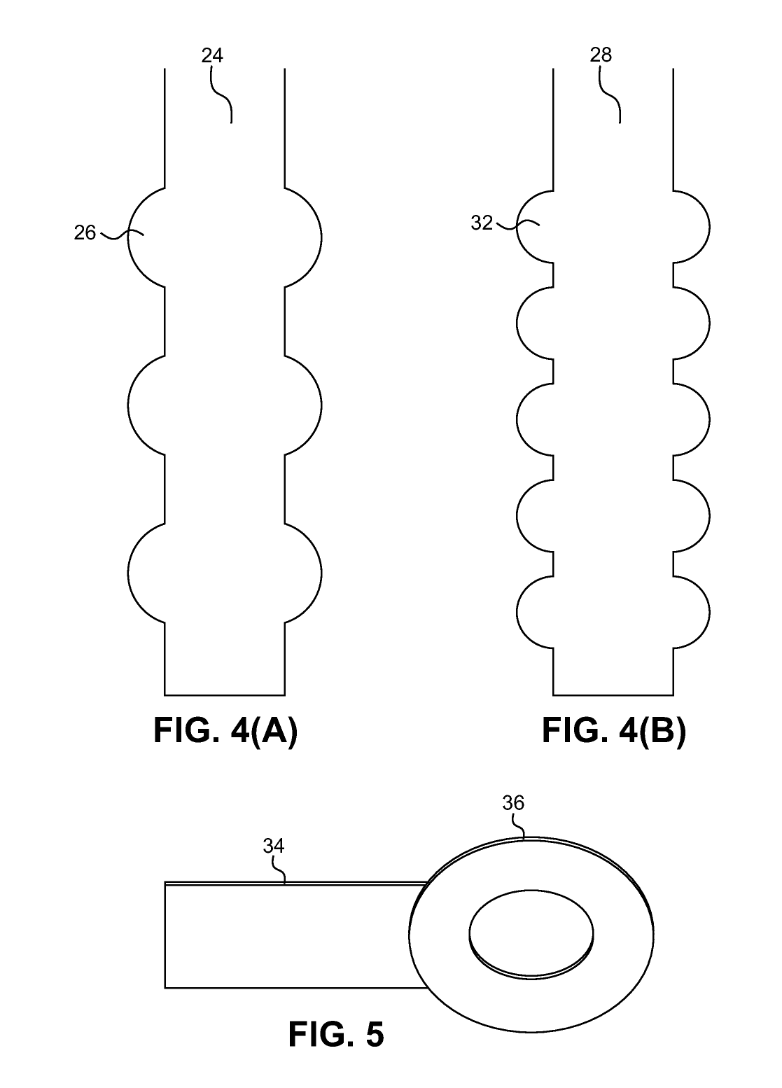

To ensure a minimum of migration, the ends of the wire or ribbon forming the ring system may be scalloped or have another shape to increase frictional or mechanical resistance against movement. Such shapes are illustrated in FIGS. 4(A)-(B). In FIG. 4(A), a distal end 24 includes scallops or ribs 26, while in FIG. 4(B) distal end 28 includes smaller but more frequent scallops or ribs 32. In addition, the external surface of the implant may have a textured surface, or may include a polymer sleeve, or a combination of the two, to further aid the device in fixation of the vessel. The polymer sleeve may also include a microcircuit to wirelessly enable electric rim interpretation during and after the procedure. Furthermore, a coating or biological agent of the implant surface may be employed to further reduce migration and/or erosion of the implant.

Referring to FIG. 5, a distal and 34 may further include a club shape 36 so as to minimize the chance of perforation.

Also referring to FIG. 5, the hole in the club-shaped end may be employed to allow two implants to be attached to each other. In this way, multiple implants may be loaded into a delivery system to allow multiple installations in a single procedure. The implants may be attached end-to-end in a way akin to staples or railcars.

The ring may employ a shoulder 18 for stability, as well as a feature 22 to cause pressure, as illustrated in FIG. 6. For example, the feature 22 to cause pressure may be any three-dimensional solid capable of exerting additional pressure along a predetermined area, such as a ridge. The portion of the shoulder adjacent to tissue may be roughened or otherwise treated in order to provide an irritant to that tissue, so as to cause endothelialization as discussed above. Such endothelial cells are typically not conductive, and thus acts as a long-term-care modality.

It is noted that limiting migration is assisted by the shape and structure of the implant device. In particular, the overall helical structure of the implant device ensures that a longitudinal force, along the axis of the device, tends to be absorbed by a compression of the helix, similar to the way in which a spring compresses, although the construction ensures that the spring constant may be extremely low, especially in the axial direction. This may be contrasted with other more stent like structures, which are designed such that a longitudinal force is transmitted along the typical chain link or honeycomb structure, causing translation or a change of radius of such structures rather than compression.

### Variations

The implant may be permanent, removable, or the same may be configured and designed to be absorbed into the body after a period of time. In a removable embodiment, a removable portion (which may be the entire implant or a portion thereof) may be installed for a period of time, e.g., between 30 minutes and 24 hours, and then removed. During this time, the device may impart pressure against the tissue, necrosing the same and rendering the local tissue electrically inert, thereby creating a block. While the procedure and device have been described in the context of the PVs, the same may be conveniently employed in the coronary sinus as well. Other potential treatment sites include the IVC, SVC, coronary sinus, and the vein of Marshall, as well as other vessels and electrically-viable substrates. In addition, the device may be employed to invoke a neurological response of the ganglion plexus.

Other implementations of the implant device may include one or more of the following. The device may include a contiguous circumferential ring substantially normally perpendicular to the ostium of the PV, and the ring or coil structure may have at least 1 full rotation, as well as a pitch that is >1° from the first coil. The ring or coil like structure may provide radial support to stenosed vessels such as a stenosed PV. The ring or coil like structure may have a distal coil and a proximal coil, the distal coil deployed at the distal end of the electrically active PV sleeve that extends within a human PV. The proximal coil may be deployed at the proximal end or ostia of the PV and employ a single extension arm or a plurality of extension arms that extend distally toward and connect to the distal ring. The extension arms may extend distally toward the distal ring in a helical pattern. The extension arms that join the distal and proximal rings may be designed to interrupt ectopic electrical signals emanating from within the PV. The ring or coil like structure may be implanted within a vessel of the heart and may generate circumferential radial pressure sufficient to block the cellular exchange of sodium and/or both sodium/calcium or potassium from entering the cell and thus rendering the cell electrically inert. The ring or coil structure may apply mechanical pressure to cardiac tissue causing focal apoptosis/necrosis. The ring or coil structure may have a material composition, surface treatment, coating, or biological agent and/or or drug to cause a human biological response, e.g., intimal hyperplasia or endothelization, in a controlled or semi-controlled way in order to effect a long-term electrical block at or within the PV or other electrically active vessels or structures within the heart. The ring or coil structure may have at least one full circumferential winding, and indeed more, and may include a helical extension moving distally from the outer diameter of the first coil and terminating within the vessel to prevent migration of the coil or ring structure. The ring or coil may be made of a round wire or ribbon profile that is shaped into a ring or coil. The ring or coil may have various cross-sectional shapes designed to focus mechanical force in a circumferential or helical pattern against the inner surface of a vessel or structure within the heart. These shapes include but are not limited to round or circular, triangular, rectangular, “U”-shaped, or any number of other shape combinations. The ring or coil may have a material composition and/or geometry designed to sufficiently conform to tissue to prevent coagulation or thrombus, and may include a material coating to further reduce or prevent such coagulation or thrombus.

In some implementations, the ring and helices may act as an electrical wave reflector, changing the course of the electrical wave back to its origin and in some implementations acting as a cancellation medium to electrical waves emanating from the source. The ring or coil structure may have a hexagonal, pentagonal, and/or octagonal shape when viewing in an end view. This geometric shape may be designed to improve conformability to the vessel following implantation. In this connection it is noted that approximately 30% of PV's have an oval shape. By changing the geometry of the loop or ring, the ring and vessel may be mutually conformed, and the radial force equalized along the circumference of the inner surface of the PVs. The ring or coil may have the above-noted shapes at the proximal end but may employ a circular shape at the distal end. The implantable devices may be employed in combination with an ICD to deliver currents or voltages to heart tissues. Such devices may be coupled to an ICD in a wired fashion or wirelessly. Other devices that may take advantage of the convenient placement of the implanted devices may similarly benefit from coupling to the same.

### Deployment

The device may be deployed in various ways.

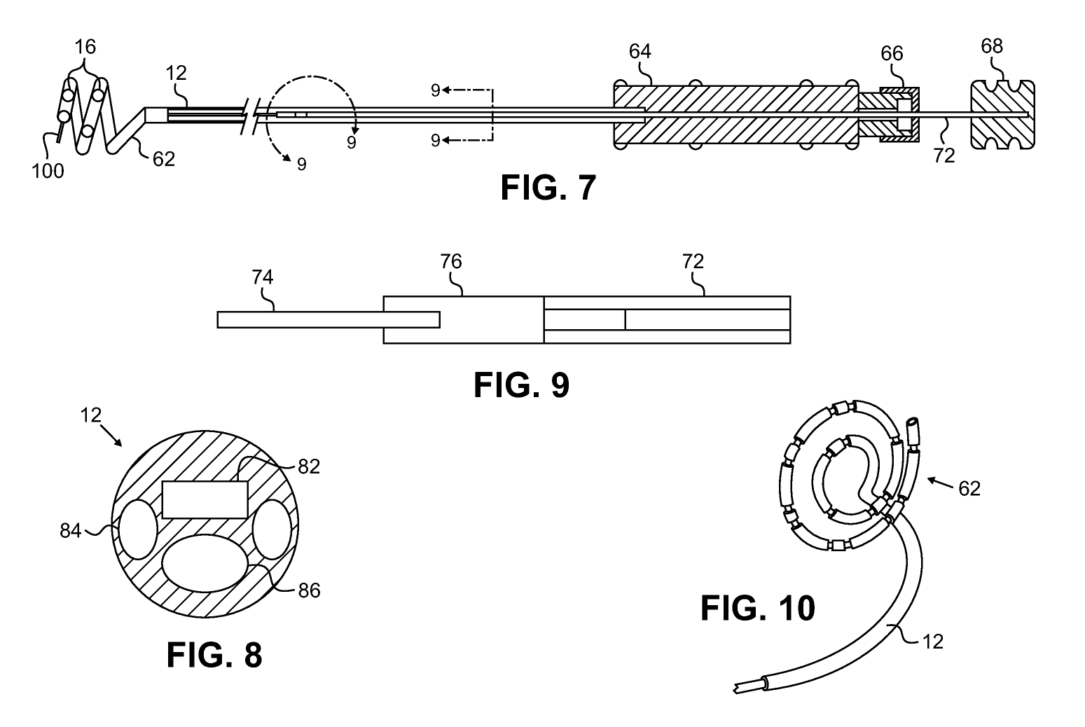

In one implementation, illustrated in FIGS. 7-10, a delivery catheter has a handle 64 for steerability and a knob 68 to control a pusher (or pushing means) 72, e.g., a flexible wire or elongated spring, at a proximal end. At a distal end, the delivery catheter may have a PeBax® (or other material) loop or pigtail 62. The pusher (shown in greater detail in FIG. 9) with a tip 76 extends through the delivery catheter 12, and the same is attached to an implant device 100 at a point within the catheter. The implant device is uncoiled in this undeployed configuration, and the implant device may extend through the pigtail 62 and may further extend a short distance from the distal end of the pigtail during deployment. The distal end of the delivery system may also include a design where the catheter distal end is in a straight or neutral position and then steered using knobs and/or levers on the handle to create the pig tail distal segment. Another lever located on the handle may be employed to deflect or steer the distal segment for cannulation of each pulmonary vein. This design may also include a plurality of electrodes to enable intra-cardiac electrogram interpretation.

By pushing the implant device out of the distal end of the catheter, shown in more detail below, the same may take up a position within the PV as desired. One purpose of the PeBax pigtail is to protect the vein during deployment in the same way, e.g., a Lasso® catheter does. In addition, the PeBax pigtail may be equipped with electrodes to allow mapping and/or ablation, as described in greater detail below. The pitch of the distal loop or pigtail may be altered in known manner, e.g., by a control wire, to allow different cardiac geometries to be accommodated. Where mapping electrodes are used, their length may range, e.g., from approximately 0.5-4.0 mm.

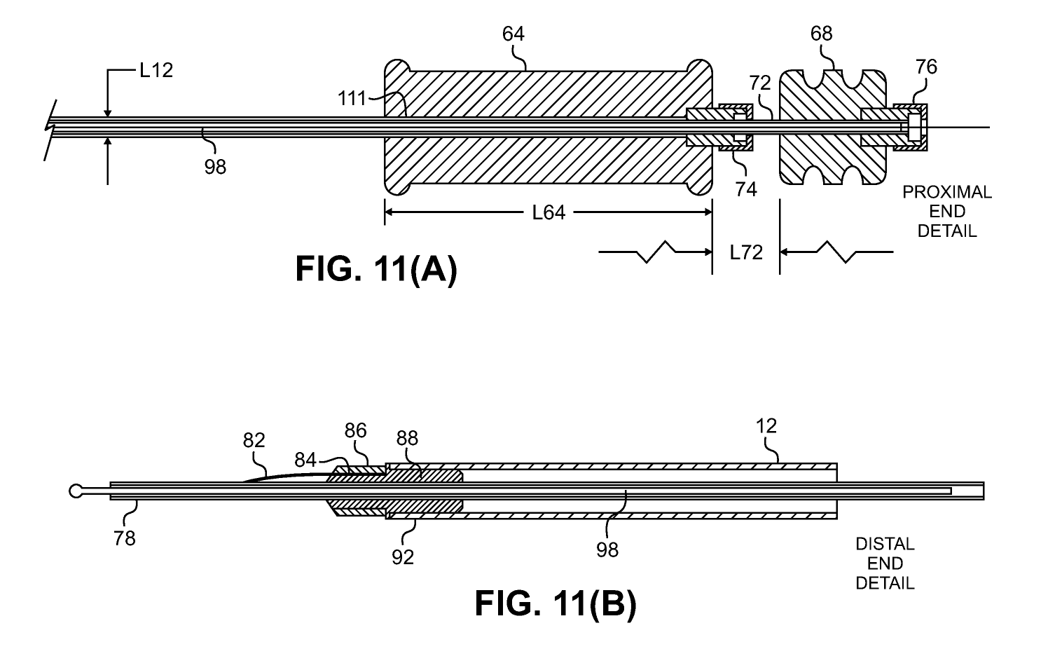

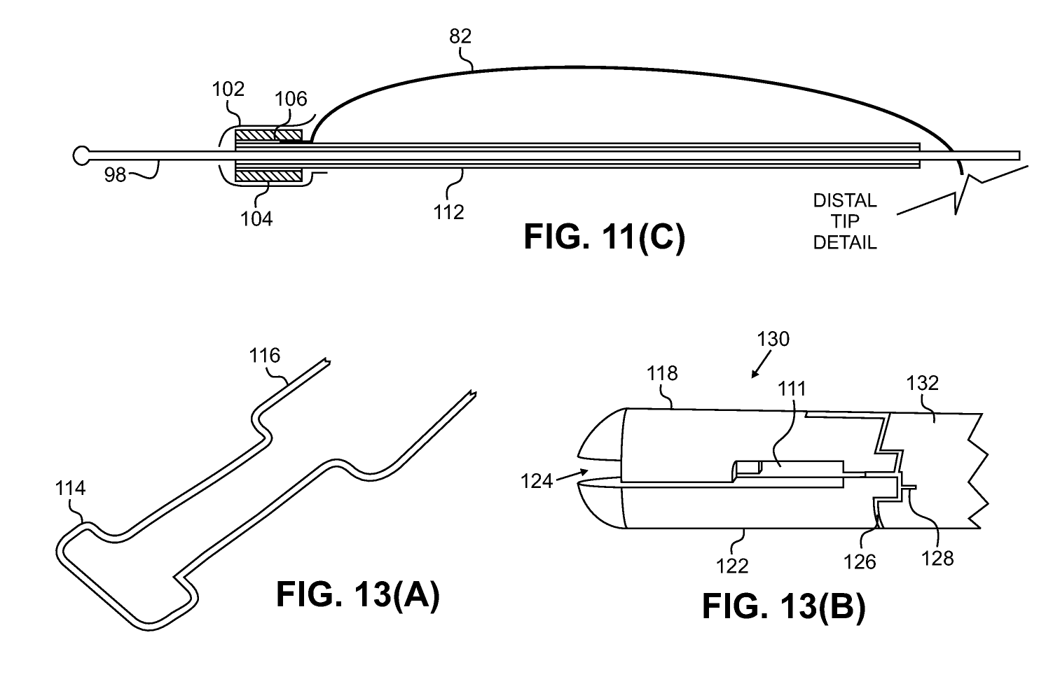

FIG. 7 also illustrates element 66, which along with elements 74 and 76 of FIG. 11(A) may constitute Tuohy-Borst hemostasis valves or adaptors.

Referring to FIG. 8, a rectangular lumen 82 may be employed to contain and deliver the implant and a circular or oval lumen 86 may be employed to contain signal wires for the mapping and ablation electrodes. Of course, it will be understood that the shape of the lumens may vary. In this way, mapping may be accomplished prior to deployment of the implant into the vein, e.g., allowing for acute block measurement. Of course, the signal block may not happen acutely in some patients, instead requiring prolonged exposure to the implant. In addition, it will be understood that more than one rectangular or circular lumens may be employed, and their shapes may differ, according to the needs of any given catheter design. In systems where the catheter is made fully steerable or deflectable, additional lumens 84 may be employed to provide the necessary control wires for steering or deflection.

FIG. 11(A)-(C) illustrate a related embodiment, as well as various construction and manufacture details of a specific exemplary version. In these figures, a handle 64 includes a knob 68 which are separated by a distance L72. The distance L72 is chosen to allow for complete deployment of the implant device. A layer of epoxy 111 may seal the handle 64 to the sheath. Referring to FIG. 11(B), the sheath 96 and seemed to terminate at a distal end at a distal end bushing 88. A hypo stock sleeve 86 surrounds a layer of epoxy 84 which is used to hold a NiTi tension band 82. The distal end bushing is coupled to the sheath 96 by a layer of epoxy 92. Referring to FIG. 11(C), greater detail is shown of the distal tip. In particular, a distal end of the NiTi tension band terminates at a hypotube 104 and is held in place by a layer of epoxy 106. A heat shrink 102 is set around the assembly.

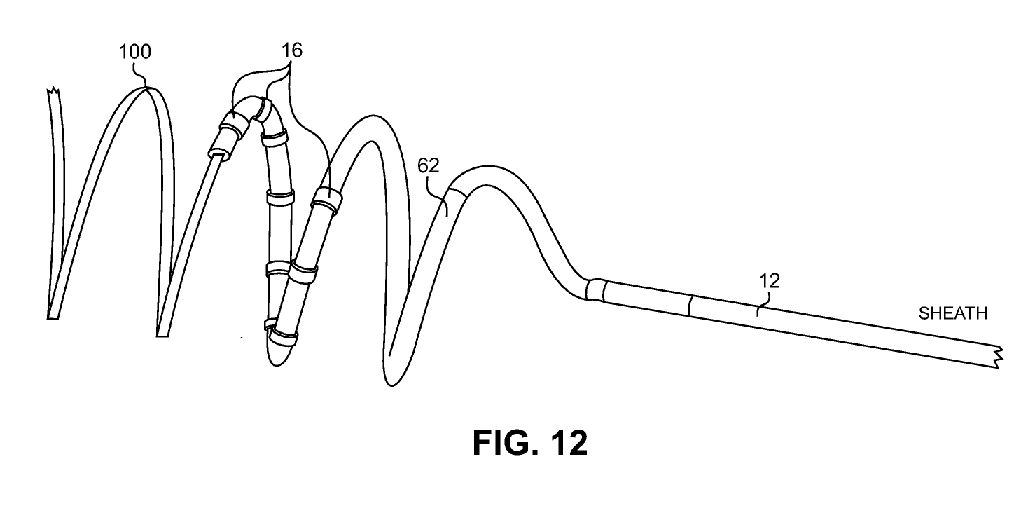

In the above implementation, and referring in particular to FIGS. 7 and 12, the design includes a spiral or pig-tail end that allows the implant to be delivered in a controlled manner and which protects the endocardial surface of the vein. The distal end of the delivery system may be employed for diagnostic purposes, such as ECG mapping of the vein prior to and after implanting the device using the electrodes 16. The distal end may also allow a user to recapture the implant using devices described below if it is partially or already deployed, enabling further control and proper placement within the PVs.

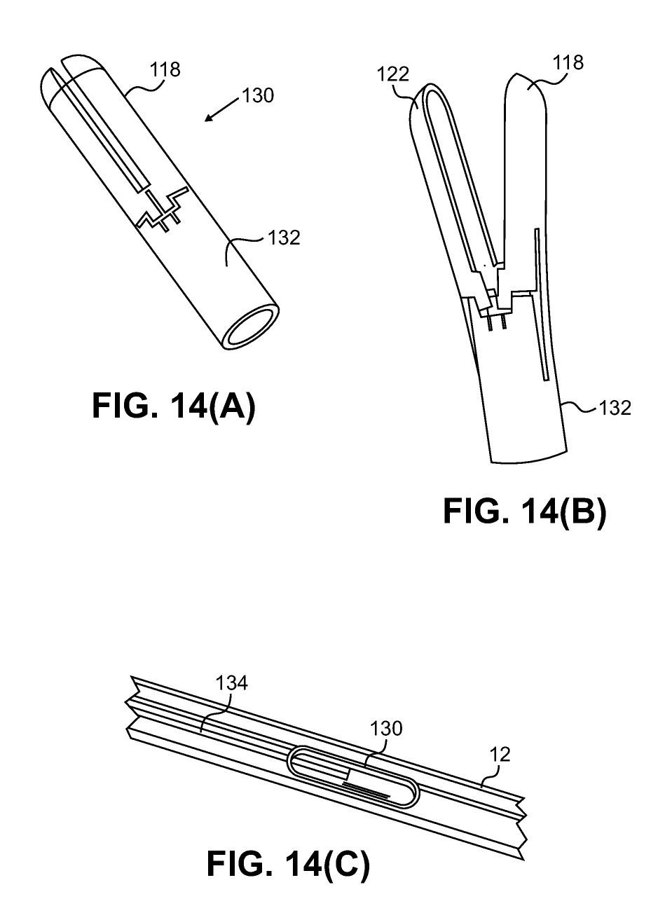

For example, referring to FIGS. 13(A) and (B) and FIG. 14, the implant may also be held by the catheter by a grabber or grip 130, e.g., a toothed grip. In particular, laser (or other) cuts 126 and 128 may be made in a distal cylindrical catheter tip to form a mouth or grip 124 which may grab the proximal end of the implant. In the figures, the laser cuts are made radially or longitudinally to the cylindrical axis of the grabber. It will be understood that curved cuts may also be employed, according to the needs of the particular application. The cuts allow bending or flexing away from the remainder 132 of the grabber or grabbing means 130. The mouth or grip may be configured, e.g., via heat treatment (e.g., using a memory metal such as nitinol) or design or both, to distend or open when the mouth or grip is not confined by the sheath tube. Once the same is thus extended away from the sheath 96, the same may open and release the implant.

In a related implementation, the implant may be formed with a groove between elements 114 and 116 (see FIG. 13(A)) or other feature to allow the grabber device 130 to hold the same in a secure and/or locked fashion. Similarly, the grabber device may have formed thereon a “tooth” 111 between upper half 118 and lower half 122 to allow additional points of contact (see FIG. 13(B)). The scalloped ends of the implant device, described above, may also be employed for this purpose.

Additional views are also shown in FIG. 14(A)-(C). In FIG. 14(C), a cutaway view of the grabber 130 is shown attached to a pusher 134 within the sheath 12.

In any case, when the grabber device navigates the sheath or delivery catheter, it must navigate both curved sections and straight sections. In some systems, it may be advantageous to provide the same with a small curve or with additional laser cuts to allow the grabber device a degree of flexibility.

Ablation with Delivery Device, Including with Partial Deployment of Implant

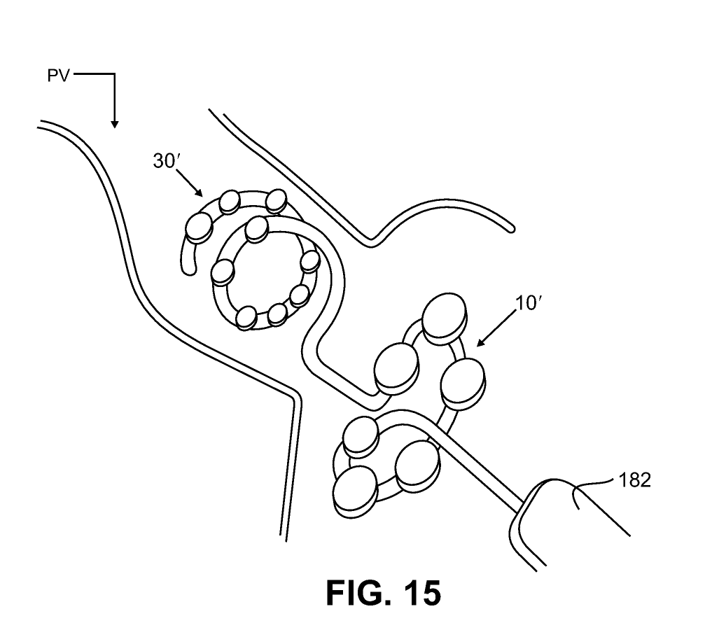

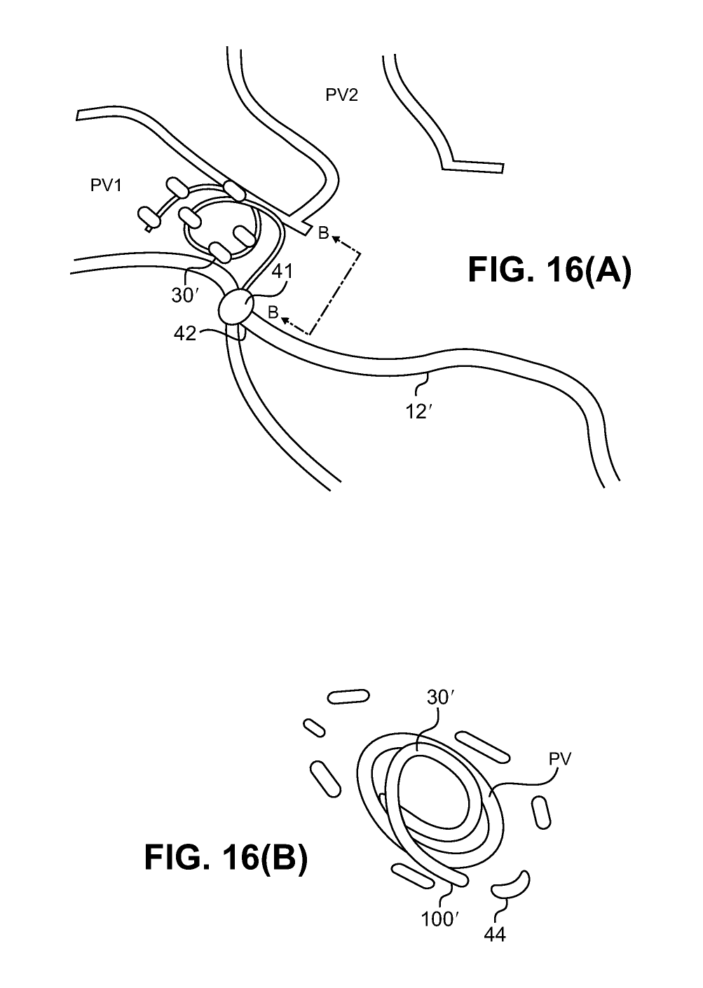

In a related device, and as shown in FIGS. 15 and 16, an ablation device may be provided with a catheter 182 coupled to a proximal ring 10′ and a distal ring 30′. The distal ring 30′ may provide both an anchoring aspect and a mapping aspect. In particular, the distal ring 30′ may incorporate a number of mapping electrodes. The proximal ring 10′ may incorporate a number of ablating electrodes. The distal set may enter into a pulmonary vein and become temporarily apposed to the inner lumen therein. In this sense, the device with two sets of electrodes may be disposed similarly to the implanted device discussed above, but in this case, the same would be retracted after treatment. The distal ring employs its electrodes for mapping, while the proximal ring may employ its electrodes for mapping and/or ablation. The apposed electrode of the distal ring may be as noted above, and while the same may become lodged with respect to translational displacement, the same may also be easily rotated with respect to a track formed by the pressure of the ring against the tissue of the pulmonary vein. The proximal ring electrodes may then contact the ostium and via RF ablation cause necrosis of a ring of tissue around the ostium. In FIG. 16(A), just one electrode 41 is illustrated, adjacent where the anchoring pigtail extends into the pulmonary vein. FIG. 16(B) also illustrates an end-on view of a device 100′, with a pulmonary vein, a distal ring 30′ within, and dashes 44 indicating the area around the ostium which is ablated. In this system, even without steering, an effective lesion may be creating by rotating the handle and ablating, resulting in a consistent and repeatable lesion that may be created safely. As the same spot is returned to in the ostium, or nearly returned to, by the electrode, or electrodes, a relatively closed-shape lesion is formed and the possibility of micro-reentrant currents is significantly reduced or eliminated. As noted above, the system may conveniently employ some of the same aspects as for the implantable ring system. For example, the cross-section of the ring, or pigtail or spiral, may be rectangular so as to result in a ribbon. A ribbon implementation provides significant translational stiffness while still allowing the system to be retracted back into a catheter. Alternatively, just a portion may be a ribbon, e.g., the distal ring, while the remainder is round, e.g., the proximal ring. Nitinol may be employed as a material for the rings. In this system, therefore, ablation may occur while mapping is also occurring simultaneously. This may be contrasted with prior systems, in which ablating, and testing the results of the ablation, must be performed serially. In this way, ablation may be stopped after a block is detected, minimizing the chance for “over-ablation”.

Of course, in the implementation of FIG. 15 it will be noted that it is not necessary for there to be two separate rings—a continuous set of electrodes may be provided, e.g., to accommodate varying sizes of vessels and cardiac features, and selective electrode activation may be employed to map and/or ablate desired tissue.

In another implementation, an implant device as described may be deployed so as to gain purchase in the PV, e.g., via a partial deployment. The electrodes on the catheter or sheath may then be revolved around the vein by rotating the handle while ablation is conducted at a plurality of locations. In this way, a well-defined circular lesion may ensue, and block may be tested for during the procedure. In this regard, it is noted that one or multiple electrodes may be activated at any one time or during any one procedure. In addition, the user can define circular lesions (by rotating the entire system) or helical lesions (but slowly extending portions of the ring device from the sheath, and revolving the sheath (but not ring device) in so doing). If multiple electrodes are activated while creating a helical lesion, then one can achieve multiple helical lesions, which have in some cases been found particularly useful for atrial fibrillation treatment.

Moreover, following ablation and/or mapping, the ring device may be fully implanted in the vein as described elsewhere. In this way, a multi-pronged technique may be employed to ensure block is achieved and maintained. Of course, in some implementations, the ring device may also be pulled back into the catheter or sheath. In this connection it is noted that the ring device may be permanently attached to the pusher.

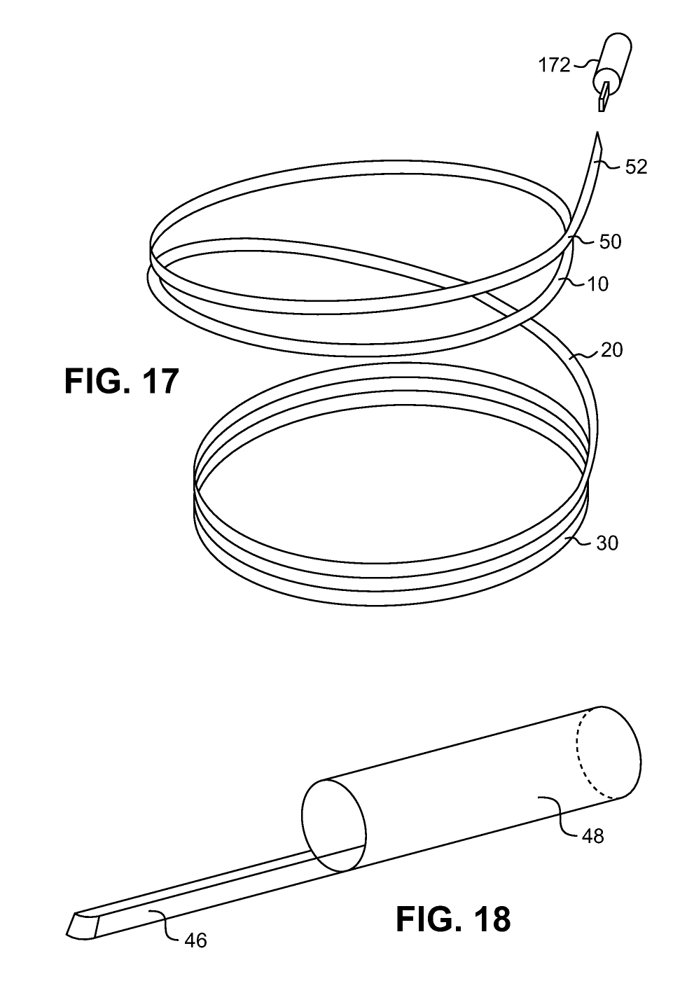

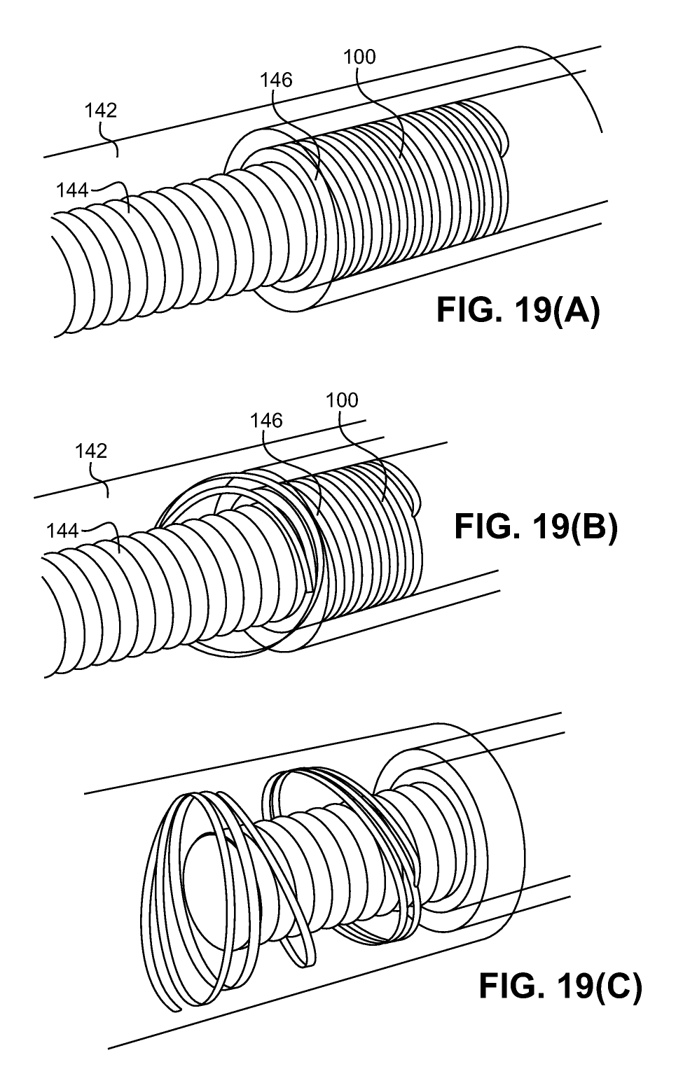

In a related implementation, as seen in FIGS. 17 and 18, the system may employ a small device, i.e., a ratchet sleeve having a cylinder 48 and extension 46, within the delivery catheter or sheath that can provide a ratcheting function. In this way, the handle may be simplified, and provided with greater control, by having the operator only have to provide a repeated short-stroke motion to controllably cause the implant to exit the sheath and become implanted in the PV.

The ratchet or ratcheting mechanism is shown in greater detail in FIG. 18 (not to scale). In particular, the ratchet sleeve is disposed within the sheath. Once the implant is pulled back into the sheath, and the ratchet sleeve is disposed near the distal tip of the sheath, then the implant may be deployed by repeatedly pushing it out of the tip, e.g., a fraction of a centimeter, e.g., a ¼ centimeter, to 2 inches, at a time. The implant is prohibited against retracting into the sheath by virtue of the ratchet sleeve.

In a further related embodiment, a small balloon may be inflated within the ratchet sleeve if desired to provide a way for the ratchet sleeve to grab onto the implant. By placing a tip of the implant, e.g., the proximal tip, into the ratchet sleeve, and inflating the balloon to fill up the interstitial space, the implant may be effectively grabbed by being held between the balloon and the wall of the ratchet sleeve. In another embodiment, the inflation lumen and balloon may be provided in the pusher, and the device may be grabbed by inserting the pusher into the ratchet sleeve and inflating the balloon, thereby constricting the implant tip in the same small diameter as the balloon (within the ratchet sleeve), causing the same to be grabbed. In yet another embodiment, a small balloon may be employed to render the volume within the ratchet sleeve closed, and in that case a small negative pressure may be pulled on the interior of the ratchet sleeve, constricting its walls and causing the same to pull inwards, grabbing onto the implant in the process.

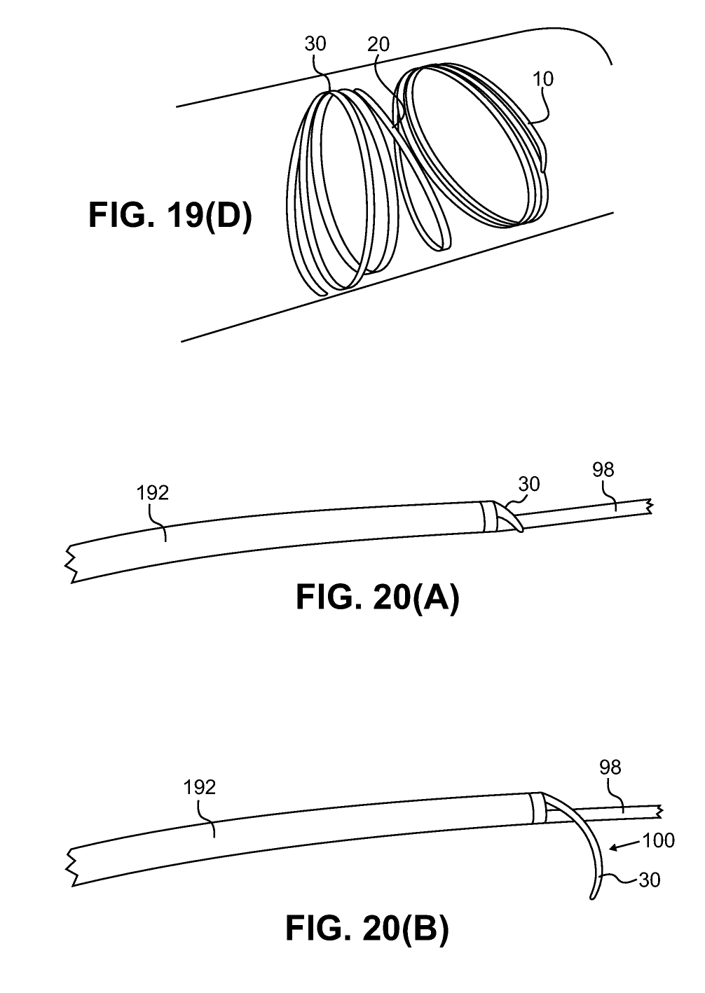

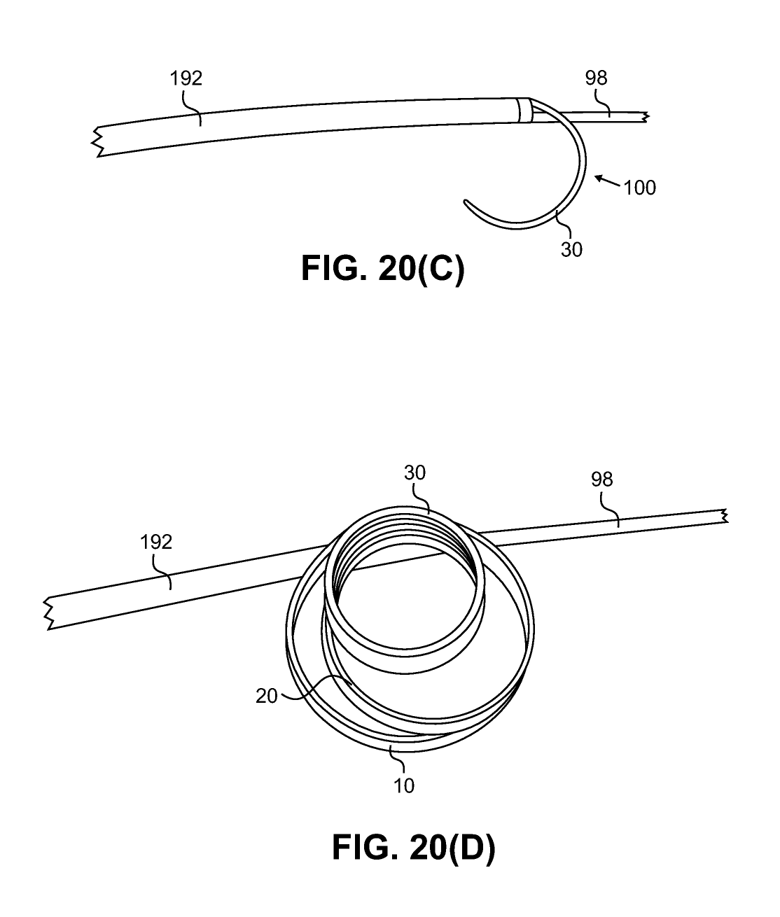

In an alternative implementation, illustrated in FIG. 19 (A)-(D), the implant device 100 is coiled around a threaded mandrel 144 and confined by an outer tube 146. Removal of the outer tube allows the implanted device to spring away from the mandrel by virtue of its shape-memory character. FIG. 19 (A)-(D) illustrates a sequence of deployment steps. In general, removing the outer tube causes immediate deployment, resulting in impingement of the device 100 against a vessel wall 142. FIG. 20 (A)-(D) illustrates another embodiment, also illustrating a sequence of deployment steps, in this case which deploys the implant perpendicularly to the direction of implantation of FIG. 19 (A)-(D). This deployment direction may be useful in certain patient anatomies. In FIG. 20 (A)-(D), the implant 100 emerges directly (and initially linearly) out of the distal tip of the catheter 192. In FIG. 20 (A)-(D), the distal ring 30 emerges first, followed by the proximal ring 10, though it will be understood that the order may be reversed.

In various implementations, the implant may be deployed from the proximal side first, such as at the ostium of the atrial/vein junction, followed by deployment of the distal ring within the vessel. The reason this is advantageous is that this can allow more mechanical force to be applied to the luminal surface of the myocardial sleeve. In particular, the first ring may be disposed in the ostial/atrial junction location, implanted, and the helices and second ring may then be unwound or uncoiled around and into the PV. This unwinding or uncoiling deployment allows installation of an implant that can provide sufficient mechanical force to achieve the clinical response necessary to create conduction block, e.g., destruction of cell coupling at the gap junction/connexin level at the intercalated disc, as well as inactivation of the Na-channels, causing dehydration of the cells by compression, resulting in conduction block. It is noted in this connection that a set of rings, connected by helical extension arms, sized for the vein, but allowed to simply expand, such as by the effect of the shape memory alloy, may in certain cases not provide the needed mechanical force to compress the surface cells. In addition, during deployment, e.g., while the implant is partially deployed, the action of the partial implant on the electrical signal propagation may be confirmed or verified to check the level of isolation achieved.

To deploy the distal end first, a split catheter shaft may be employed, such that separation of the catheter shaft at a location near the distal end causes the distal end to be deployed first. Of course, in certain implementations, the proximal end may also be deployed first. Such a split catheter shaft may be employed, e.g., in the delivery of the implant shown in FIG. 19 (A)-(D). In this implementation, the distal end of the catheter may employ a polymer tip for atraumatic delivery, and the polymer tip may be radiopaque. As in most of the implementations described, the catheter may be delivered over a guide wire.

In another implementation, the distal end of the device is sutured to the catheter, and the wire of the device is wrapped around the catheter. In this connection it is noted that the implant, during delivery, undeployed and constrained in a delivery device, may take the form of a straight wire, a helically-wrapped wire, or another configuration. The sutured end causes the distal end to be deployed last, and the final separation of the distal end from the catheter may be effected by way of cutting using a blade configured for that purpose, an electrical arc, or the like.

In general, the delivery system will have distal and proximal ends, where the distal end employs an atraumatic distal tip and the proximal end includes a handle. The system further includes a catheter shaft having a tubular structure traversing from the proximal end to the distal end. The guidewire lumen includes a luminal space to enable passage of a range of guidewire sizes. In one implementation, the guidewire lumen is furthermore capable of being advanced distally or proximally to enable deployment of the coil-like implant attached along the external surface of the guidewire lumen and contained within the inner surface of the outer catheter shaft. As in some embodiments above, the delivery system catheter may employ a flexible distal segment and a steering wire anchored at the distal portion of the delivery catheter.

In some implementations, the deployment device, or another device, may allow a degree of recapture to occur in order to fix incorrect implanted device placements within the PV. For example, where the device is pushed through a tube for deployment, the same two may be used to deliver a small wire equipped with maneuverable jaws at its distal end (such as are shown above in various embodiments). In some cases, for example, a modified guide wire may be employed. A control wire running alongside the guide wire may allow the contraction of one or more jaws in order to grab an errant device. If desired, retraction of the guide wire may then allow the complete removal of the implanted device. In the system described above where a mouth or grip is closed or opened by virtue of its being enclosed by a sheath or not, respectively, the mouth or grip may be employed to recapture an implanted device. In the same way, the ratchet sleeve with incorporated balloon may provide this function as well.

Multiple ring devices may be delivered in a single surgical operation, such as in the four pulmonary veins in a given patient. For example, in such a procedure, MRI may be employed initially in order to determine sizes of the various pulmonary veins. According to the order the physician intends to use for deployment, suitable rings may then be loaded into the device. For example, the physician may intend a plan of treatment in a clockwise direction starting with the left superior pulmonary vein, followed by the left inferior pulmonary vein, followed by the right inferior pulmonary vein, followed by the right superior pulmonary vein. The device efficacy may then be verified by performing a pacing and mapping maneuver in each vein. That is, conduction block may be verified following deployment, such as by using the mapping capability described in this specification. It is believed to be a particularly beneficial advantage that multiple device deployment and verification may be achieved using a single “stick” through the septum. The above procedure of deployment may only require, e.g., 15 to 20 minutes.

### Mechanism of Operation

Both rings as well as the helix or helices may compress tissue, stopping the propagation of aberrant signals associated with atrial fibrillation in a manner disclosed below. This compression is not to necrose tissue; rather, the same is to cause a narrowing of certain channels within the tissue associated with the propagation of aberrant electric signals. For example, sodium, calcium, or potassium channels may be blocked by mild compression. It is believed that a suitable amount of force will result in a compression of the first one to five cellular layers in the tissue. In particular, it may be important to at least compress the first layer. Using such a device and method, PV isolation may be achieved without means of an energy source or surgical procedure.

It is believed that the amount of pressure necessary should be more than 10 grams per square millimeter, e.g., greater than 20 grams per square millimeter, but less than 340 grams per square millimeter, e.g., less than about 200 grams per square millimeter, as noted above. While it may be desired to have the rings and helix or helices exert a relatively constant force around the circumference of the vein, it is more likely, given anatomical imperfections, that certain areas will receive more pressure than others. However, compliance of the ring and the use of the helix helps to distribute forces around the implant. In general it is believed that the amount of pressure needed will primarily be a function of the material used, the diameter of the artery or vein, and the thickness of the muscle sleeve.

It is noted that the distal ring, inside the PV, as well as the helices, may perform an anchoring function as well as a conductive block function. Moreover, it is noted that a full conductive block is not necessary, nor is full transmurality needed. In some cases, merely a slowing down of the net signal propagation may be enough to frustrate the arrhythmia. For example, a 50% conduction slowing may be highly significant in stopping the propagation of aberrant signals. In any case, the device's geometry, roughly matching the myocardial sleeve, further enhances this effect. It is noted in this connection that throughout the length of the PV, ‘hot spots’ exist where ectopic beats may originate. If the configuration of the ring is such that these are disrupted, then the disruption can act as an efficacious treatment per se. Such disruptions may be particularly effected by the helices between the rings. It is also noted that the ring inside the PV allows for a therapeutic treatment modality in the vein but without the serious complications associated with prior RF or cryogenic in-the-vein treatments, or the like.

It is also noted that the ring may cause the vessel in which it dwells to become more oval or round, or otherwise to maintain a more open shape than that which it adopted before, in the absence of the implant. In this way, the device acts as a stent, enhancing hemodynamics and the resulting blood flow. The device affects the shape of the vein, and vice-versa. This effect improves apposition of the implant to improve outcomes by enabling circumferential contact resulting in conduction block, laminar blood flow, and can help to treat stenotic vessels. One aspect of the device that assists in this regard is the device ring compliance, which causes the device to conform to the vessel—i.e., the radial expansion helps to keep the device in place in a dynamic way, which current PV stents generally cannot. In some cases, the device may be specifically installed to perform the function of a PV stent, and if used in this way, generally, a double-helix design may be employed between the two rings.

It is noted that the above channel-blocking effect of the implant has a multi factorial response mechanism. First is an acute response that, depending on implementation, may last from 1-45 days. After this, depending on the degree to which the implanted device has been treated, a secondary biological or chronic response mechanism may ensure long term block as a result of the biological response to the implant, e.g., endothelialization, the same starting at 15-30 days and lasting indefinitely. The biological response of endothelization cell proliferation is designed to replace myocardial cells or the cells that conduct electrical conduction with endothelial cells that are incapable of electrical cell-to-cell conduction. The treatment of the device refers to, e.g., the level to which the device has been roughened so as to act as an irritant to the adjoining tissue. The amount of endothelialization may be ‘tuned’ by this degree of roughening, which may occur via bead blasting, etc. The treatment may also be via surface modification, coatings, or the like.

In some implementations, the metallic nature of the implanted device may be employed to provide a level of active heating so as to heat or necrose tissue adjoining the implant. For example, such heating may be by way of induction using a device external to the patient. The device may be caused to heat the implant and thus heat (and treat) the tissue creating localized necrosis, and then be easily removed from the vicinity of the patient to stop the heating. In advanced versions of this implementation, the heating device and the implant may be tuned such that only one implant is heated at a time, if multiple implants have been deployed.

### Construction

As will be understood, the rings and helices may be constructed of several types of materials. For example, biocompatible metals such as nitinol may be employed, and the same exhibit useful shape memory properties. Biocompatible polymers or elastomers may also be employed.

If the ring is made of materials that are bioabsorbable, then the same may eventually be absorbed into the PV by virtue of the endothelialization, leaving only (and at most) a scar visible on the inside of the PV.

### Coatings

While not required in all implementations, various coatings or other agents may be applied or made part of the rings and/or helices, such coatings or agents capable of disrupting the propagation of aberrant electrical signals or otherwise treating arrhythmias. Such coatings may include drugs, biologics, chemicals, or combinations, and the same may cause some degree of necrosis that by itself or in combination with the mechanical compression acts as a treatment for arrhythmias. For example, a coating including alcohol may be employed as a sort of chemical ablation reagent. Such coatings may also enhance endothelialization as discussed above. As another example, the rings and helices may be coated with tantalum, e.g., a 3-5 micron coating.

Various illustrative implementations of the present invention have been described. However, one of ordinary skill in the art will recognize that additional implementations are also possible and within the scope of the present invention. Accordingly, the invention is to be limited only by the claims appended hereto.

1. A implant device for treating a malady, comprising:
a. a proximal ring;
b. a distal ring; and
c. an extension arm connecting the proximal ring to the distal ring.

2. The implant device of claim 1, wherein the extension arm includes at least one helical winding.

3. The implant device of claim 1, wherein the proximal ring and the distal ring include coils of a ribbon.

4. The implant device of claim 3, wherein a radius of the proximal ring is greater than the radius of the distal ring.

5. The implant device of claim 3, wherein each coil includes at least one winding of the ribbon.

6. The implant device of claim 5, wherein each coil includes at least 1.5 windings of the ribbon.

7. The implant device of claim 3, wherein each coil includes a pressure feature.

8. The implant device of claim 7, wherein the pressure feature is a ridge.

9. The implant device of claim 1, wherein in an undeployed configuration the radius of the proximal ring is between about 4 to 60 mm and the radius of the distal ring is between about 6 to 60 mm.

10. The implant device of claim 9, wherein in a deployed configuration the radius of the proximal ring is between about 2 to 40 mm and the radius of the distal ring is between about 3 to 40 mm.

11. The implant device of claim 1, wherein the rings are configured to deliver a force against adjacent tissue when deployed of between about 5 g/mm² and 340 g/mm².

12. The implant device of claim 11, wherein the rings are configured to deliver a force against adjacent tissue when deployed of between about 20 g/mm² and 200 g/mm².

13. The implant device of claim 1, wherein the proximal ring is configured to deliver a lesser force when deployed against adjacent tissue than the distal ring.

14. The implant device of claim 3, wherein the width of the ribbon is between about 0.5 and 2.5 mm.

15. The implant device of claim 14, wherein the width of the ribbon is between about 1 and 2 mm.

16. The implant device of claim 1, wherein an extremity of the ring is shaped to increase frictional or mechanical resistance against movement.

17. The implant device of claim 16, wherein the extremity is shaped to include scallops, ribs, or a club shaped end.

18. The implant device of claim 1, wherein the implant device is coated with a material composition, surface treatment, coating, or biological agent and/or drug.

19. A kit for treating a malady by deploying an implant device in a vessel, comprising
a. the implant device of claim 1; and
b. a delivery system, the delivery system including a catheter having a pigtail distal end,
c. such that upon deployment of the implant device from the pigtail distal end, a longitudinal axis of the implant device is substantially collinear with a longitudinal axis of the vessel.

20. A method for treating a malady, comprising:
a. choosing a size of an implant device for insertion into a vessel of a patient, the implant device including a proximal ring, a distal ring, and an extension arm connecting the proximal ring to the distal ring; and
b. inserting the implant device into the vessel of the patient,
c. such that the choosing includes selecting the size of the implant device such that the implant device compresses a K, Ca, or Na channel in adjacent tissue sufficiently to block or to delay electrical signals traveling along the axis of the vessel.

21. The method of claim 20, wherein the inserting includes delivering the implant to the vessel through a catheter including a pigtail distal end.

22. The method of claim 20, wherein the vessel is a pulmonary vein.

23. The method of claim 22, further comprising mapping at least one pulmonary vein.

24. The method of claim 22, further comprising of ablating at least one pulmonary vein.

25. The method of claim 24, wherein the ablating is performed using at least one electrode disposed on a delivery device.

26. The method of claim 20, wherein the inserting includes delivering the distal ring into the pulmonary vein and delivering the proximal ring into the os of the pulmonary vein.

27. The method of claim 20, further comprising administering local anesthesia and not general anesthesia to the patient.

28. The method of claim 21, wherein the inserting further includes pushing the implant device through the catheter with a pushing means.

29. The method of claim 28, wherein the pushing means is coupled to the implant device using a grabbing means.

30. The method of claim 23, wherein the mapping includes determining the sizes of at least two pulmonary veins, and further comprising delivering at least one implant device to each pulmonary vein.

31. The method of claim 30, further comprising loading implant devices into the delivery device in the order in which they are to be successively implanted in pulmonary veins.

32. The method of claim 20, wherein the malady is atrial fibrillation or vessel non-patency.

33. The method of claim 20, further comprising inducing a local heating effect to be present on the implant device by induction.

34. The method of claim 20, further comprising recapturing the implant device after the inserting.

35. The method of claim 20, wherein the compression of the K, Ca, or Na channel in adjacent tissue sufficiently to block electrical signals traveling along the axis of the vessel includes compressing the first one to five cellular layers of the adjacent tissue.

36. The method of claim 23, wherein the mapping is performed both before the inserting and after the inserting.

37. The method of claim 20, wherein the compression is such that the delay is caused in conduction of at least 50%.

38. A method for treating a malady, comprising:
a. choosing a size of an implant device for insertion into a vessel of a patient, the implant device including a proximal ring, a distal ring, and an extension arm connecting the proximal ring to the distal ring; and
b. inserting the implant device into the vessel of the patient,
c. such that the choosing includes selecting the size of the implant device such that the implant device causes a necrosis in adjacent tissue sufficient to block electrical signals traveling along the axis of the vessel.

39. A method for treating a malady, comprising:
a. choosing a size of an implant device for insertion into a vessel of a patient, the implant device including a proximal ring, a distal ring, and an extension arm connecting the proximal ring to the distal ring; and
b. inserting the implant device into the vessel of the patient,
c. such that the choosing includes selecting a radius of the distal ring of the implant device to be at least two times the radius of the vessel.

40. The method of claim 39, further comprising selecting a radius of the distal ring of the implant device to be at least five times the radius of the vessel.

41. A method for treating a malady, comprising:
a. inserting a catheter into a vessel of a patient, the catheter having loaded within an anchoring device for partial insertion into a vessel of a patient, the anchoring device including at least a distal ring;
b. partially extending the distal ring from the catheter such that the distal ring is anchored in the vessel;
c. activating at least one electrode on the catheter, the at least one electrode substantially adjacent to tissue when the distal ring is anchored in the vessel, the activating causing ablation and necrosis of the adjacent tissue;
d. retracting the distal ring into the catheter; and
e. withdrawing the catheter.

42. The method of claim 41, further comprising activating a plurality of electrodes on the catheter, the electrodes distributed along the pigtail distal end.

43. The method of claim 41, further comprising rotating the catheter at least partially during the activating, thereby causing ablation and necrosis of tissue and the creation of partial circumferential linear lesions.

44. The method of claim 41, further comprising inserting an implant device into the vessel, the implant device including a proximal ring, a distal ring, and an extension arm between the proximal and distal ring.
